(19) United States
(12) Patent Application Publication
(10) Pub. No.: US 2008/0017205 A1
(43) Pub. Date: Jan. 24, 2008

(54) Method and Device for Releasing Flavor and Odor Substances

(76) Inventor: Ahron Chazot, Emek HaYarden (IL)

Correspondence Address: BROWDY AND NEIMARK, P.L.L.C.;624 NINTH STREET, NW, SUITE 300, WASHINGTON, DC, 20001-5303, US

(21) Appl. No.: 10/591,648

(22) PCT Filed:
(86) PCT No.: PCT/IL05/00252

(30) Foreign Application Priority Data
Mar. 4, 2004 (IL) 160753

(51) Int. Cl. A24F 47/00

(52) U.S. Cl. 131/270

(57) ABSTRACT

A device for releasing substances into the mouth, that comprises an intra-oral pacifier provided with a sucking element that partially or fully occupies the mouth of a user. The sucking element is used for releasing flavor substances and is made from materials in the natural or unnatural latex family. An adequate amount of the substances is provided inside, or on the pacifier. The substances may include some nicotine for smoking cessation, antibacterial materials for curing intra-oral diseases, or flavor or odor substances.

### FIELD OF THE INVENTION

The present invention relates to the field of flavor and/or odor releasing devices, which can be substitute devices for smoking. More particularly, the invention relates to a device for releasing flavor, odor, antibacterial and curing substances and to a substitute adapted for enabling a gradual withdrawal from dependency or addiction to smoking.

### BACKGROUND OF THE INVENTION

Flavor, taste, and an appetizing appearance are primary factors in a consumer's decision to buy a product. Volatile colorants and aromas can be stabilized and their processing can be made simpler through microencapsulation. Microencapsulation technology provides viable texture blending, appealing aroma release, and taste-, odor-, and color-masking, whereby generating layers. In microencapsulation, capsules are designed and prepared to meet all the requirements in due consideration of the core material, intended use of the product and the environment of storage. Microencapsulated products can be divided into five main categories: (a) flavorings; (b) vitamins and minerals; (c) oils and fats; (d) herbs and bioactives; and (e) other food ingredients. In case, when microencapsulated products comprise nicotine or compounds synthesized from said nicotine, these products can be used for smoking cessation.

Smoking tobacco products, whether in the form of cigarettes, cigars, or pipes, are detrimental and hazardous to a person's health. Numerous well-publicized studies have indicated that the incidence or risks of lung cancer, emphysema and cardiac problems is significantly higher in heavy smokers than in non-smokers, and that the incidence and effects increase with the extent of smoking. Many studies also indicate, if the diseases have not yet started, or some are not far advanced, that much of the risk can be eliminated by stopping or drastically curbing the smoking habit.

In spite of the apparent conclusiveness of such studies, a large percentage of the population continues to smoke. Many persons who do smoke recognize those inherent risks but feel the present satisfactions gained by smoking outweigh possible future bad effects or find that they are addicted to smoking to the extent that they are unable to stop.

Various means and methods have been devised for trying to break a smoker's dependency or addiction to tobacco. Some of those involve ingesting of materials that cause extreme discomfort when the person later smokes. Others involve other unpleasant stimuli applied when the person smokes. The concept of such methods is to provide negative associations with smoking.

Still other methods suggest substitutes for smoking. For example, when a person craves a cigarette or cigar, he chews a stick of gum or eats fruit instead. Special compounds have been disclosed to act as substitutes for tobacco, for example, in U.S. Pat. No. 107,693 or U.S. Pat. No. 1,474,110.

A problem with such substitutes or unpleasant associations is that they hardly reduce or eliminate a smoker's addiction to tobacco. Often, this addiction is stronger than the satisfaction of the substitutes, or the tobacco withdrawal symptoms are more unpleasant than the unpleasant associations.

As a result, the cessation of smoking is often only temporary; many smokers have “quit” smoking dozens of times, each time having returned to smoking to satisfy their craving for tobacco addiction. Often too, a heavy smoker undergoes undesirable personality changes while attempting to stop, and consequently is often encouraged by others to return to smoking to regain his former personality. Still others, by substituting eating for smoking rapidly gain weight upon stopping smoking and soon start smoking again to lose weight. It is thus apparent that such methods are not satisfactory, particularly for heavy smokers who have smoked for a long time.

Such other methods, exemplified by the disclosures of U.S. Pat. No. 3,877,468 and U.S. Pat. No. 3,901,248, provide a chewable smoking substitute that contains nicotine. By chewing the substitute, a person obtains the nicotine craves, or is addicted to without smoking; hence, the smoking habit can purportedly be broken. However, the nicotine in such chewable smoking substitute fades away relatively quickly, and the chewable element becomes useless.

It is an object of the present invention to provide a smoking substitute for overcoming some of the problems and shortcomings of the prior art.

It is another object of the present invention to provide a smoking substitute to be used by a user for a relatively long period of time.

It is yet another object of the present invention to provide a pleasantly flavored smoking substitute which aids smoking cessation or reducing the amount of smoke consumption.

It is a further object of the present invention to provide a relatively comfortable and discrete smoking substitute for the user.

It is still a further object of the present invention to provide a device for releasing flavor and odor substances.

It is still a further object of the present invention to provide a device for releasing antibacterial materials or substances for curing intra-oral diseases.

Other objects and advantages of the invention will become apparent as the description proceeds.

### SUMMARY OF THE INVENTION

The present invention relates to flavor and/or odor releasing devices, which can be substitute devices for smoking.

A device for releasing substances into the mouth comprises: (a) an intra-oral pacifier provided with a sucking element, partially or fully occupying the mouth of a user, wherein the sucking element is used for releasing flavor substances and is made from materials in the natural or unnatural latex family; and (b) an adequate amount of said substances provided inside or on said pacifier.

Preferably, the substances include an adequate amount of nicotine and are used for smoking cessation.

Preferably, the device further comprises antibacterial materials or substances for curing intra-oral diseases.

Preferably, the device further comprises flavor or odor substances.

Preferably, the mixture of natural or unnatural added flavorings includes sugars, flavor, odor, antibacterial materials for curing diseases.

Preferably, the sucking element further comprises at least one hole along its body for allowing a user to take in or push out air through said hole, thereby refreshing the intra-oral cavity and/or increasing the activity of intra-oral materials of the user with the pacifier.

Preferably, the sucking element can be provided in the form of any desired shape and size suitable to be inserted into or placed in the mouth or intra-oral cavity of a user.

Preferably, the materials are rubber or silicone with natural or unnatural flavorings which include nicotine or flavor, odor, antibacterial materials or curing substances.

Preferably, the sucking element comprises predetermined amount of dextrorotary isomer of nicotine uniformly dispersed in the material(s) comprising dosage of substitute nicotine.

Preferably, the sucking element is coated with flavored materials.

A method for releasing substances comprises: (a) providing an intra-oral pacifier, occupying at least a part of the mouth of a user comprising a sucking element made from materials in the natural or unnatural latex family; (b) providing an adequate amount of said flavor substances with said pacifier; and (c) causing said sucking element to release said substances while being in the user's mouth.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 schematically illustrates intra-oral pacifiers for aiding smoking cessation, according to a preferred embodiment of the present invention;

FIG. 2 schematically illustrates the intra-oral pacifiers of FIG. 1 provided with a groove for the teeth, according to a preferred embodiment of the present invention;

FIGS. 3A and 3B schematically illustrates the intra-oral pacifiers of FIG. 1 located within the mouth of a user.

### DETAILED DESCRIPTION OF PREFERRED EMBODIMENTS

The invention will now be described primarily with reference to the examples of device for withdrawal from addiction to smoking. These examples are provided merely to illustrate the invention and are not intended to limit its scope in any way. It should be understood that the invention can be applied mutatis mutandis to other types of addiction, such as addiction to food consumption. Furthermore, the invention can be used for releasing flavor, odor, antibacterial materials or substances for curing intra-oral diseases.

The present invention relates to a flavor and/or odor releasing device and to a smokeless device which is used as a substitute for smoking. The smokeless device is an intra-oral pacifier, preferably—but not limitatively, having a form of an elliptic or rounded bead or other similar shape suitable and fitted to be used in the mouth of a user. Of course, it is most desired that the shape and size of the intra-oral pacifier will be adapted to provide maximum comfort to the user while using said intra-oral pacifier. As the intra-oral pacifier is intended to be used within the intra-oral cavity of the user, it can be used in a discrete manner.

FIG. 1 schematically illustrates an intra-oral pacifier for releasing flavor and/or odor substances and for aiding smoking cessation, according to a preferred embodiment of the present invention. Pacifier 10 comprises a sucking element 11 made from materials in the natural or unnatural latex family, including silicone, which has a releasing flavor capability. The natural or unnatural latex based pacifier 10 is provided with an adequate amount of nicotine and/or other taste producing flavors in any form. The mixture of natural or unnatural added flavorings can also include sugars, such as fructose or any other supportive additives.

Pacifier 10 has at least one hole or perforation 12 along its body in order to allow a user to take fresh air in or out of the mouth, through perforation 11, thereby refreshing the intra-oral cavity and/or increasing the activity of intra-oral materials of the user with the pacifier.

Preferably, the sucking element 11 can be in the form of any desired shape and size suitable to be inserted into or placed inside the mouth of the user.

The material from which the sucking element 11 is made can be other improved latex products comprised of natural or unnatural latex (e.g., rubber or silicone) with natural or unnatural flavorings which could include nicotine and/or other supportive additives, such as sugar.

According to a preferred embodiment of the present invention, the pacifier 10 comprises a predetermined amount of dextrorotary isomer of nicotine uniformly dispersed in the material(s) comprising a dosage of substitute nicotine.

According to a preferred embodiment of the present invention, the pacifier 10 is provided with a groove or notch (not shown) for easily holding the pacifier with the teeth during intake or output of air via the hole or perforation 12.

It should be noted, that according to all embodiments of the present invention, pacifier 10 can be provided with flavor, odor, antibacterial, curing and other types of substances. For example, if a user has some kind of wound in his tongue, instead of putting a sticker, he can use pacifier 10 releasing antibacterial and curing substances during the sucking process. These substances disinfect intra-oral cavity and prevent possible infection of the user's mouth. For example, if the user has a headache, he can use pacifiers 10 which can slowly release substances preventing said headache. Furthermore, pacifier 10 can be used by a baby who was born to a parent addicted to drugs. In this case, pacifier 10 should comprise active substances decreasing the suffering of said baby. According to another embodiment of the present invention, pacifier 10 can comprise teeth whitening and protecting materials.

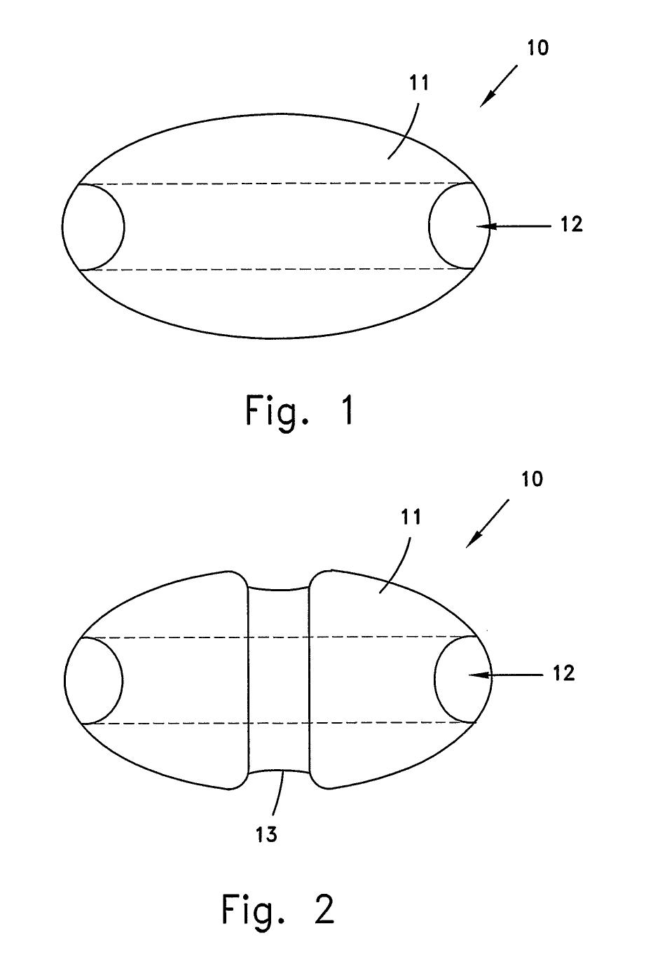

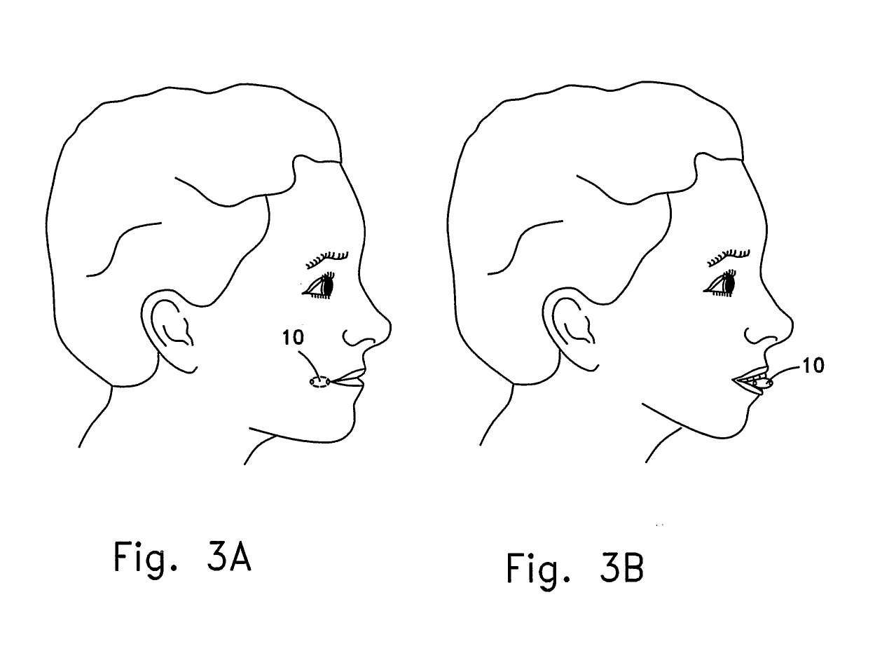

FIG. 2 schematically illustrates the intra-oral pacifiers 10 provided with a groove 13 for the teeth. FIGS. 3A and 3B show the use of pacifier 10 by a user. In FIG. 3A, the pacifier 10 is located within the mouth of the user. In FIG. 3B the pacifier 10 is located between the teeth of the user.

Nicotine is a colorless, oily liquid having the chemical formula of C10H14N2, formally referred to as 1 methyl-2-(3-Pyridyl) pyrrolidine and is present in tobacco to the extent of 1.8 percent. Nicotine is also a molecular asymmetrical organic compound; as such, it exhibits optical activity, i.e., nicotine has the property of causing rotation of the plane of polarized light passing there through.

As derived from tobacco, nicotine is a levoratory (l-) compound, causing plane polarized light passing there through to rotate to the left. Nicotine may, however, also be chemically synthesized in a manner known to those skilled in the art, and from the synthesized compound, a dextrorotary (d-) nicotine isomer or compound may be isolated.

Both the tobacco derived l-compound or isomer and the synthesized d-compound or isomer have identical chemical formulas (C10H14N2), have the same chemical reactions, and are alike in physical properties. However, they differ in physiological action and effect. Stated otherwise, the l- and d-isomers have different effects on living organisms. Accordingly, it has been found and reported that the synthesized d-isomer of nicotine is only one half as toxic as the natural tobacco-derived l-isomer.

This difference in toxicity of the l- and d-isomers is very important in formulating smoking substitutes and in formulating those in a manner in which they can be used to gradually diminish and eliminate a person's dependence on smoking, particularly since both isomers otherwise give the same effect and sensations, to the same degree, to a person. Thus, a given amount of the synthetic d-isomer of nicotine gives a person the same sensations and satisfaction as a like amount of the natural, l-isomer nicotine, but at only half the toxicity, or at the same level of toxicity, the d-isomer provides greater effects and satisfaction to a user than does the l-isomer.

Several results follow from the reduced toxicity of the d-isomer. More of the d-isomer nicotine can safely be used in a smoking substitute such as the intra-oral pacifier 10 of the present invention, than of the l-isomer nicotine; consequently, a substitute having greater user satisfaction can be provided with no greater, or even less, toxic reaction. Secondly, because a greater amount of the d-isomer nicotine can be used in the smoking substitute, normal tolerances in compounding the substitute have relatively less effect. Thirdly, because the d-isomer nicotine can be used in the smoking substitute, without increasing toxicity, it is possible to more easily and practically formulate a graduated series of dosages having decreasing amounts of nicotine, so that over a period of time a person can be gradually weaned from smoking without experiencing withdrawal symptoms.

The amount of nicotine used in the pacifier 10 should be similar to the amount of nicotine used in typical or other existing substitute devices, such as a chewing gum, as is known to a person skilled in the art. For example, the percentage of weight of l-isomer nicotine relative to the base material (e.g., silicone) used varies from about 0.05 percent to 2.0 percent, and the percentage weight of the d-isomer nicotine preferably varies from about 0.02 percent to 8.0 percent. Whereas the weight per dosage of the l-isomer has been disclosed to vary from about 1 to 10 milligrams, the weight per dosage of the d-isomer may vary from about 0.5 to 40 milligrams.

Use is possible with the d-isomer of nicotine, the only requirement being that a relatively high latex base concentration be used. Such concentration should be at least about 40 percent by weight of the latex as ready for use.

Other commonly used additives, such as defatted cocoa, flavoring and colorings may also be used. Sugar, in the form, for example, of sucrose or corn syrup may also be added.

Preferably—but not limitatively, in order to delay release of all of the d-isomer nicotine, the isomer is bound to a cation exchanger of the type described in U.S. Pat. No. 3,901,248. By binding the isomer to a cation exchanger, absorption of the isomer in the mouth is reduced and more of the isomer is carried to throat region, to provide more of the sensations associated with smoking, since for optimum effectiveness the smoking substitute should closely approximate those associated with smoking.

Actual formulation of latex for sucking element 11, exclusive of the d-isomer of nicotine and the cation exchanger, to which it may be bound if desired, is well known to those persons skilled in the art and consequently need not be described herein in detail, it being understood that the d-isomer, as well as the cation exchanger, can be used with substantially any natural or unnatural latex process.

The smoking substitute pacifier 10, according to the present invention can be made most conveniently by a mixture of a conventionally compounded latex base or mass with the d-isomer or with the d-isomer cation exchanger. The d-isomer or d-isomer exchanger are conveniently added to the latex mass along with the addition of other conventional additives such as sugars or flavorings. Depending on the properties of the acidifying agent, if one is to be used, it will be found convenient, for example to add liquid acidifying agents, for example, sulphuric acid, with conventional liquid additives such as corn syrup. Non-liquid acidifying agents, for example, malic acid, may be added with conventional non-liquid additives, such as powdered sugar.

Preferably, also, the d-isomer or d-isomer exchanger is added when the latex mass has cooled to an extent that the mass is just mixable, in order to prevent losses of the isomer which may occur at high temperatures.

After such additives and mixture, the sucking element 11 is formed into oval or other shapes suitable to be used within the mouth of a user, in any conventional manner, as is known to a person skilled in the art. The weight of each sucking element 11 is preferably that of conventional sweet or chewing gum, i.e., between about 1.0 to 3.0 grams. The weight percentage of the d-isomer, compared to the weight of the latex base is preferably in the range of about 0.02 to 8.0 percent, the weight of the d-isomer in each sucking element 11 being in the range of about 0.5 to 10 milligrams.

An effective manner to enable withdrawal of a person from dependency upon, or addiction to, smoking is by forming the pacifier 10, in such a manner that some sucking elements 11 have a relatively high concentration of the d-isomer and others have a lower concentration. Thus a kit or package is provided in which some sucking elements 11 have, e.g., about 16 to 20 milligrams of the d-isomer, others have, e.g., about 12 to 16 milligrams, others about 8 to 12 milligrams and so forth until some have only 1 to 4 milligrams. The sucking elements 11 are color, size, shape, or otherwise coded in a manner enabling ready identification of at least the relative amounts of the d-isomer present. For example, a sucking element 11 having about 20 milligrams of the d-isomer may be colored green, those having about 16 milligrams may be colored red, and so forth.

When starting to use the pacifier 10, a sucking element 11 having the highest amount of the d-isomer is sucked e.g., for several days or a week. Then, over a period of time, sucking elements 11 with gradually decreasing amounts of the d-isomer are used, the user being gradually withdrawn from the dependency upon smoking without experiencing withdrawal symptoms.

The intra-oral pacifier of the present invention can be used by a user for a relatively long period of time, as the pacifier is directed to be sucked or played in the mouth, rather than being chewed. By not chewing the intra-oral pacifier the effect of the nicotine lasts longer.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A device for releasing substances into the mouth, comprising:
a. an intra-oral pacifier provided with a sucking element, partially or fully occupying the mouth of a user, wherein the sucking element is used for releasing flavor substances and is made from materials in the natural or unnatural latex family; and
b. an adequate amount of said substances provided inside or on said pacifier.

2. Device according to claim 1, wherein the substances include an adequate amount of nicotine and are used for smoking cessation.

3. Device according to claim 1, further comprising antibacterial materials or substances for curing intra-oral diseases.

4. Device according to claim 1, further comprising flavor or odor substances.

5. Device according to claim 1, in which the mixture of natural or unnatural added flavorings includes sugars, flavor, odor, antibacterial materials for curing diseases.

6. Device according to claim 1, in which the sucking element further comprising at least one hole along its body for allowing a user to take in or push out air through said hole, thereby refreshing the intra-oral cavity and/or increasing the activity of intra-oral materials of the user with the pacifier.

7. Device according to claim 1, in which the sucking element can be provided in the form of any desired shape and size suitable to be inserted into or placed in the mouth or intra-oral cavity of a user.

8. Device according to claim 1, in which the materials are rubber or silicone with natural or unnatural flavorings which include nicotine or flavor, odor, antibacterial materials or curing substances.

9. Device according to claim 1, in which the sucking element comprising predetermined amount of dextrorotary isomer of nicotine uniformly dispersed in the material(s) comprising dosage of substitute nicotine.

10. Device according to claim 1, in which the sucking element is coated with flavored materials.

11. A method for releasing substances, comprising:
a. providing an intra-oral pacifier, occupying at least a part of the mouth of a user comprising a sucking element made from materials in the natural or unnatural latex family;
b. providing an adequate amount of said flavor substances with said pacifier; and
c. causing said sucking element to release said substances while being in the user's mouth.

12. Method according to claim 11, wherein the pacifier is further comprising an adequate amount of nicotine and is used for smoking cessation.

13. Method according to claim 11, further comprising releasing antibacterial materials or substances for curing diseases.

14. Method according to claim 11, further comprising releasing flavor or odor substances.

15. Method according to claim 11, wherein the mixture of natural or unnatural added flavorings includes sugars, flavor, odor, antibacterial materials or substances for curing diseases.

16. Method according to claim 11, wherein the sucking element further comprising at least one hole along its body for allowing a user to take in or put out air through said hole, thereby refreshing the intra-oral cavity and/or increasing the activity of intra-oral materials of the user with the pacifier.

17. Method according to claim 11, wherein the sucking element is provided in the form of any desired shape and size suitable to be inserted into or placed in the mouth or intra-oral cavity of a user.

18. Method according to claim 1, wherein the materials are rubber or silicone with natural or unnatural flavorings which include nicotine, flavor, odor, antibacterial materials or substances for curing diseases.

19. Method according to claim 11, wherein the sucking element comprising predetermined amount of dextrorotary isomer of nicotine uniformly dispersed in the material(s) comprising dosage of substitute nicotine.

20. Method according to claim 11, wherein the sucking element is coated with flavored materials.
